(19) United States
(12) Patent Application Publication
(10) Pub. No.: US 2017/0333618 A1
(43) Pub. Date: Nov. 23, 2017

(54) PORTABLE UV-C SYRINGE SANITIZER

(71) Applicants: Eric Robert Krohn, Binghamton, NY (US); Wayne Kerber, Binghamton, NY (US)

(72) Inventors: Eric Robert Krohn, Binghamton, NY (US); Wayne Kerber, Binghamton, NY (US)

(21) Appl. No.: 15/468,254

(22) Filed: Mar. 24, 2017

Related U.S. Application Data
(60) Provisional application No. 62/339,984, filed on May 23, 2016.

(51) Int. Cl. A61M 5/00; A61L 2/10; A61L 2/26

(52) U.S. Cl. CPC A61M 5/001; A61L 2/26; A61L 2/10; A61L 2202/11; A61M 2205/8206; A61M 2209/084; A61L 2202/24

(57) ABSTRACT

A portable syringe sanitizer kit has a longitudinal housing having a sanitization area, a compartment for retaining at least one source of electricity, and mechanisms for retaining a needle, barrel, and plunger of a syringe. A source of germicidal radiation, such as a UV-C light, is disposed in the housing, the source of germicidal radiation being electrically connected to a source of electricity, such as one or more batteries. A switch operatively connected to the source of electricity and to the source of germicidal radiation can initiate activation of the radiation. A syringe holder tube and a mechanism for retaining a syringe plunger are also provided. The source of germicidal radiation generates light having a wavelength of between approximately 200 nm and 280 nm. The housing is mounted in an eyeglass case. A timing device de-energizes the source of radiation after a predetermined interval of time.

### RELATED PATENT APPLICATION

The present application is related to copending provisional patent application No. 62/339,984, for PORTABLE UV-C SYRINGE STERILIZER filed May 23, 2016, and hereby incorporates the teaching therein by reference.

### FIELD OF THE INVENTION

This invention relates to sterilization appliances for use by end-users of syringes and, more particularly, to an appliance that utilizes a UV-C radiation in between syringe use to aid in sanitization prior to reuse.

### BACKGROUND OF THE INVENTION

The invention generally relates to sterilization appliances for use by end-users of syringes. Specifically the invention is involved with utilizing a UV-C radiation with a wavelength between 255 and 300 nm, and preferably about 280 nm, in between syringe use to aid in their sanitization prior to reuse.

When a person uses a syringe, a small quantity of blood is drawn into the syringe to determine if the needle has been properly located in the vein. Even when person then injects all of the needle's contents, a small residue of blood remains in the needle and syringe, which may not be visible. If a second person then uses this same needle and syringe for an injection, any residue in the syringe and needle may be transferred into the second user.

The goal of the present invention is to provide an inexpensive route for sanitizing syringes between uses with the goal of preventing cross contamination of blood. While current practices of utilizing bleach and filters aid in deactivating viruses, they are still unable to reach all the locations that blood is being deposited within the syringe and may not be readily available. Without making a radical change in the construction of a syringe, which undoubtedly would increase their cost, an active route must be found. The inventive sanitizer incorporates a germicidal UV-C light to aid in deactivating viruses found on the plunger and needle of the syringe. Inventors of the past have utilized UV-C light in various forms of utensils, however they were not designed for the personal syringe user to aid in virus/disease prevention.

The electromagnetic wave spectrum ranges from cosmic to radio. The UV region of the electromagnetic spectrum is situated between visible light and x-rays. The full UV radiation spectrum ranges from wavelengths of approximately 100 nanometers (nm) to 400 nm with the UV radiation spectrum being divided into 4 sections; vacuum UV radiation (100 nm to about 200 nm), UV-C radiation (called germicide or short-wave UV, 200 to 280 nm), UV-B radiation (called mid-wave, 280 nm to 320 nm), and UV-A radiation (black-light, or long wave UV, 320 nm to 400 nm).

UV radiation can be produced by sources such as light emitting diodes (LEDs), lasers, electric arcs, xenon bulbs, halogen bulbs, excimer bulbs, and mercury vapor lamps/tubes of low, medium and high pressures. UV-C radiation is used extensively for sterilization, purifying, and deodorizing applications in food, air, fluid, and general surface sterilization processes because the UV-C radiation deactivates micro-organisms such as: bacteria, molds, spores, fungi, and viruses.

Hepatitis C virus (HCV) is a small enveloped, positive-stranded RNA virus classified within the family Flaviviridae, genus Hepacivirus. HCV affects an estimated 170 million people worldwide and is a global health problem. Unlike most RNA viruses which usually cause acute diseases, HCV establishes life-long, persistent, intrahepatic infections in a majority of infected individuals, leading frequently to the development of cirrhosis and hepatocellular carcinoma. HCV is transmitted primarily via percutaneous exposure to infectious blood. HCV is capable of person-to-person transmission of via blood-contaminated objects and medical devices. UV light irradiation is a commonly used physical method for viral inactivation. UV-C with a wavelength range of 200-280 nm prevents viral replication by inducing formation of pyrimidine dimers in the viral genome.

Currently, injection of illicit drugs represents a major risk and a growing epidemic. It has been determined that HCVcc in culture medium (2.5×10⁴ FFU/ml, volume depth of 0.2 cm) could be inactivated completely by UV-C irradiation at a dose of 2.7×10⁻² J/cm² within 1 min. Therefore, UVC light irradiation represents a highly effective means for inactivating HCV.

### Description of Related Art

Certain patents and published patent applications teach similar design to the subject invention, but their use is not for the individual syringe user, contained in a non-stigma forming case and/or battery powered.

U.S. Patent Application No. 2010/0320405 filed by Gardner III and published on Dec. 23, 2010 for HANDHELD PORTABLE MULTI PURPOSE STERILIZING WAVELENGTH TRANSFORMING CONVERTER, describes a handheld portable multi-purpose device, for producing multiple and variable wavelength distributions of UV radiation, or visible radiation, comprising a primary UV radiation source and a system of wavelength transforming (WT) materials that allows selecting between UV A, UV B, and UV C radiation (individual selections or various combinations) and visible radiation.

U.S. Pat. No. 6,787,782 describes a system that utilizes UV radiation emitting LEDs as one source of UV radiation for sterilizing air in a vehicle. Similarly, U.S. Pat. No. 6,333,748 describes the use of UV radiation emitting LEDs in combination with a reactive surface to sanitize air for breathing inside a helmet.

U.S. Pat. No. 6,172,657 issued to Kamakura, et al. for BODY MOUNT-TYPE INFORMATION DISPLAY APPARATUS AND DISPLAY METHOD USING THE SAME issued on Jan. 9, 2001, describes a display unit attached to the head of a user and a control circuit unit attached to the waist or the shoulder. The display unit enables a light beam from the outside to enter the eyes of the user. Another light beam represents an image produced by the control circuit unit. The user can accordingly observe the image including required pieces of information for working together with the outside view. Available input means include magnetic and optical pointing devices and a CCD camera for reading a bar code. The control circuit unit that is not attached to the body may be connected with peripheral equipment and used as a desktop computer.

U.S. Pat. No. 4,952,369 issued to Belilos for ULTRAVIOLET DEVICE AND ITS USE issued on Aug. 28, 1990 describes an ultra-violet flashlight comprising a housing containing an ultra-violet light source, a power source, an on/off switch and a gravity switch, and a method for instantly sterilizing objects with which a person comes in contact comprising passing over said objects ultra-violet radiation from said flashlight.

World Patent Publication No. WO/2016/001776 by Messina, et al. for DEVICE FOR THE STERILISATION OF STETHOSCOPES published Jan. 7, 2016 describes a device for sanitizing medical instruments, in particular stethoscopes. The device comprises a case, a command and control unit, and a battery. The device comprising means for coupling to a stethoscope to be sanitized or sanitized, the coupling means being magnetic or electromagnetic or a mechanical coupling means. The outside of the case has a recess which forms an inverted cone, the base of which is open and substantially at the level of the outer surface of the case.

### SUMMARY OF THE INVENTION

In accordance with one aspect of this invention, a plastic housing is attached to an “eyeglass case” using glue or other suitable adhesive. The plastic housing contains electronic circuitry, a UV-C bulb, batteries, and syringe supports. A UV-C bulb producing a wave length of approximately 275 nm is utilized along with commonly found batteries for power. The syringe rests in a holder with the plunger and needle thereof being inserted into a sanitization unit, which is covered with a clear window for safe inspection. The UV-C bulb is powered on by the press of a power button and operates via a timer for a set time interval.

### BRIEF DESCRIPTION OF DRAWINGS

A complete understanding of the present invention may be obtained by reference to the accompanying drawings, when considered in conjunction with the subsequent detailed description, in which:

FIG. A is a schematic view of a syringe, showing the parts thereof;

FIG. 1 is a perspective view of the portable syringe sanitizer with the top cover removed, in accordance with the present invention;

FIG. 2 is an exploded view of the inventive sanitizer shown in FIG. 1;

FIG. 3 is top, cut-away view of the sanitizer shown in FIGS. 1 and 2; and

FIG. 4 is a cross-sectional view of the sanitizer shown in FIGS. 1, 2, and 3.

Like reference numerals refer to like parts throughout the several views of the drawings.

### DETAILED DESCRIPTION OF THE PREFERRED EMBODIMENT

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

Referring now to FIG. A, a conventional syringe 5 is shown. Syringe 5 has a body 6, a needle 7 extending therefrom, and a plunger 9 surrounded by a barrel 8.

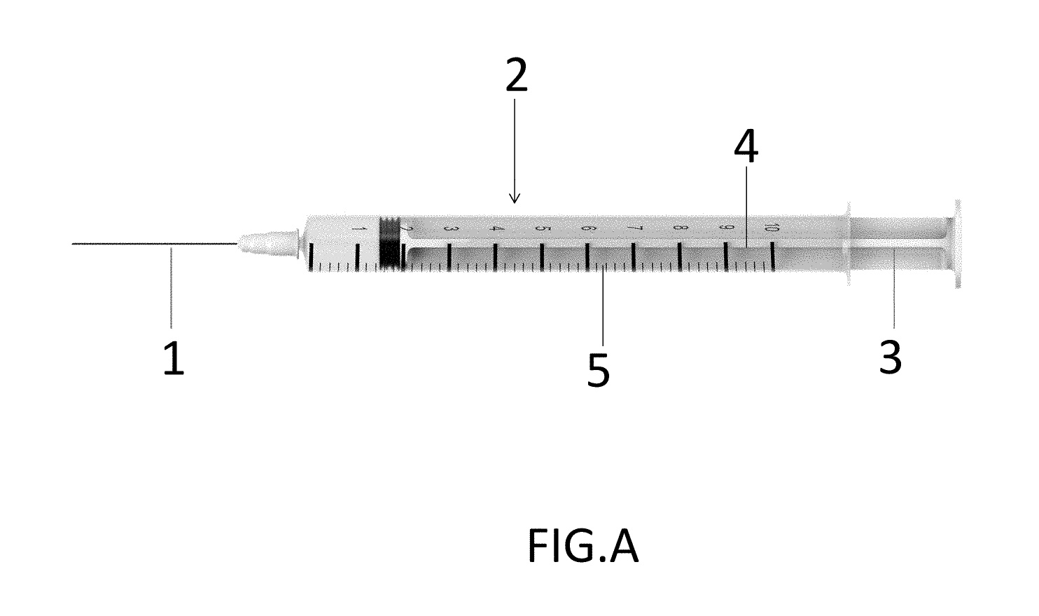

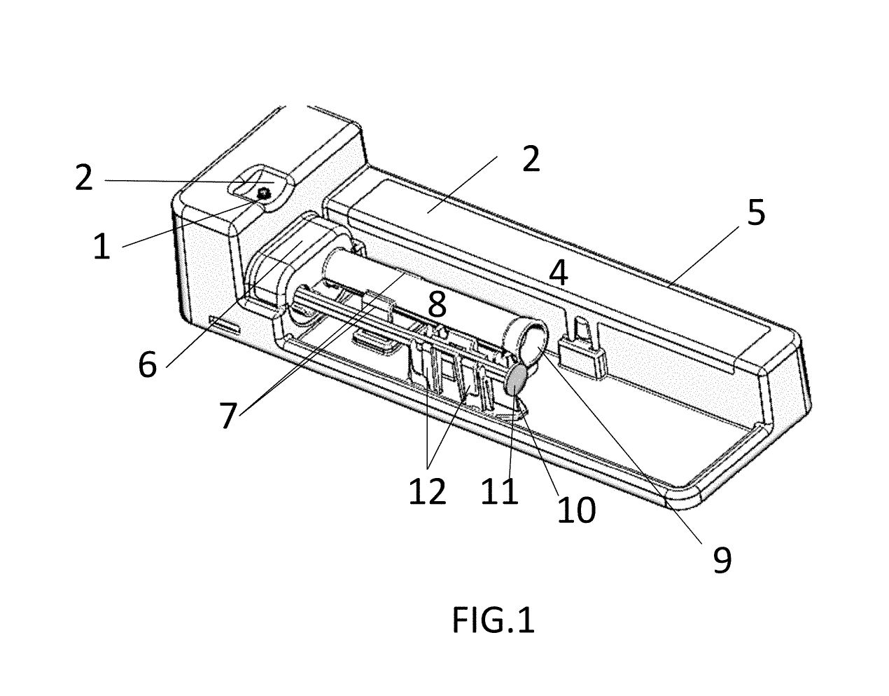

Referring to FIG. 1, a longitudinal housing 10 contains all the parts of the invention. Housing 10, in the preferred embodiment, is formed from plastic, but other suitable materials can be used. In the final product, housing 10 is inserted and attached into an eyeglass case, not shown, by suitable adhesive. Disposed in housing 10 and constrained by oppositely disposed clamps or brackets 12 is a syringe holder 14 in the form of a longitudinal tube or barrel. Syringe holder 14 is flared at the distal end 16 thereof. Brackets 12 are fastened to the lower portion of housing 10 by any one of a number of techniques well known in the art. The components of syringe 5 are held firmly to housing 10 to prevent damage and ensure sanitization efficacy.

Parallel to syringe holder 14 and spaced apart therefrom is space for a plunger 18 to be secured to housing 10 by guides 20. The distal end of plunger 18 has a flat, circular plate 22 attached thereto. Parallel to syringe holder 14 is a battery compartment 26 and cover 28 associated therewith.

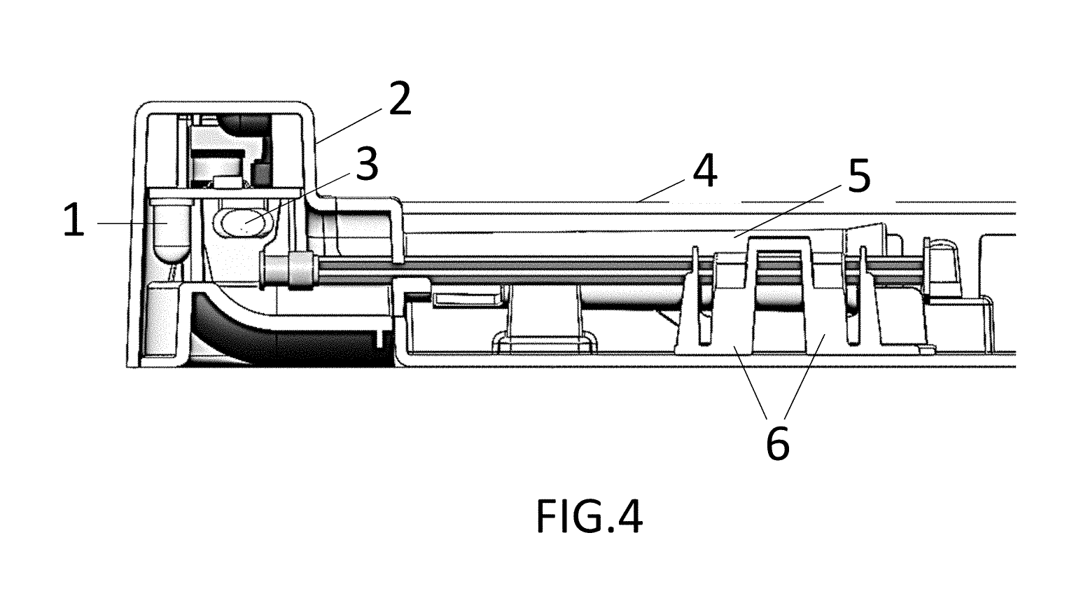

The proximal ends of syringe holder 14 and plunger 18 are inserted into a sterilization unit 24 having apertures formed therein to retain the respective proximal ends of syringe holder 14 and plunger 18. Sterilization unit 24, fabricated from translucent plastic in the preferred embodiment, allows a user to see if a “working light” 23 (FIG. 4) is on, the working light 23 being supplied by an LED. This feature provides both a way to know if the sanitizer has power and the unit is still working. Sterilization unit 24 contains a UV-C bulb 25 and a reflector 36 fabricated from aluminum or some other reflective material that may be coated with aluminum spray. UV-C bulb 25 is preferably Model No. ______ LED, manufactured by the ______ Company, but other sources of UV light can be used. Also contained in sterilization unit 24 is electrical circuitry 27 for activating UV-C bulb 25. Electrical circuitry 27 also has a timing mechanism to de-energize UV-C bulb 25 after a predetermined time interval from initial energizing.

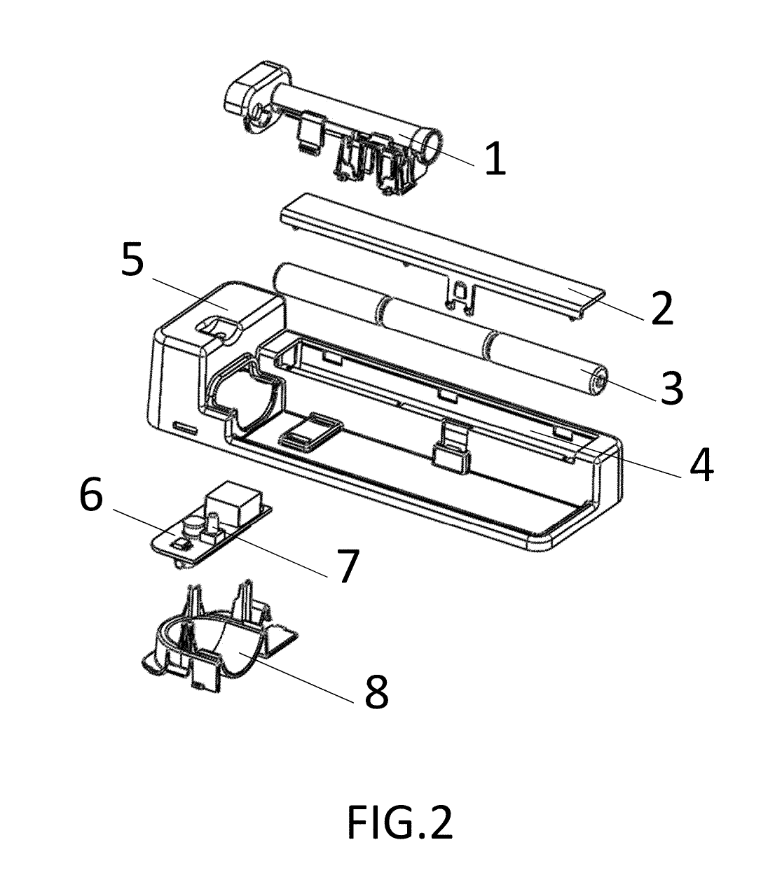

A power button 30 is disposed in an indention 32 of housing 10. Power button 30 is operatively connected to batteries 33 (FIG. 2) and electrical circuitry 27 for initiating energization of UV-C bulb 25 and timing mechanism.

Referring now to FIG. 2, batteries 33 are shown in relation to batter compartment 26 and cover 28.

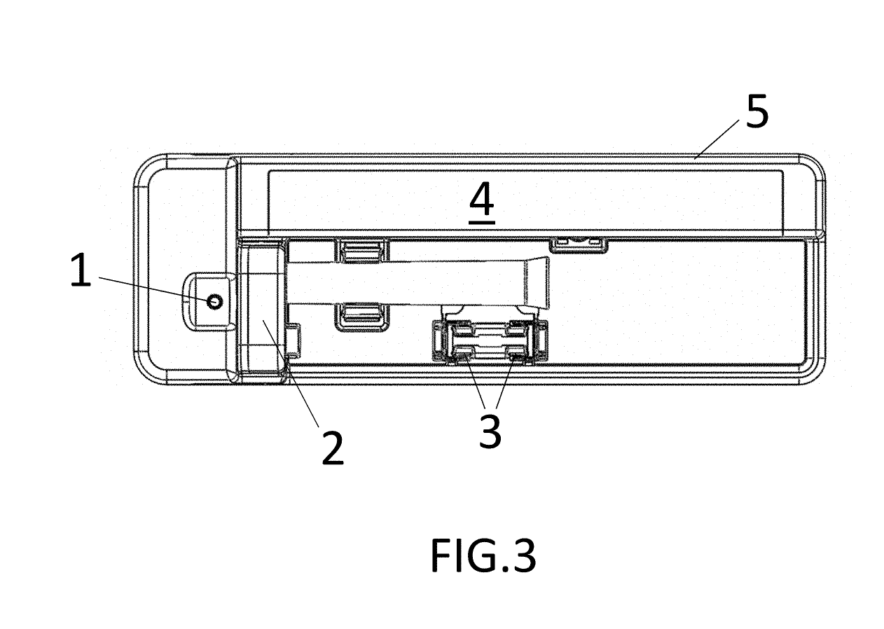

FIG. 3 is a top view of the inventive sanitizer showing placement of battery compartment 26, syringe holder 14, plunger retaining guides 20, sterilization unit 24, and power button 30.

FIG. 4 is a cross-sectional view of the inventive sanitizer showing working light LED 23, UV-C bulb 33, sterilization unit 24, syringe holder 14, and plunger retaining brackets 20.

In operation, a user separates a syringe 5 into its needle/barrel portion 8 and its plunger portion 9. Batteries 33 are placed into battery compartment 26 to provide power to the electrical components. In the preferred embodiment, three size AAA 1.5 v batteries 33 are used to power the unit, but it should be noted the invention is not limited to the number, size, or quantity of batteries.

Needle/barrel 7, 8 slides snugly into the sheath portion of syringe holder 14. Plunger 9 snaps into the place for plunger 18 in housing 10 after plunger portion 9 enters sterilization unit 24.

When power button 30 is pressed, electrical circuitry 27 and components connected thereto are powered from batteries 33 and remain on for a predetermined interval, five minutes in the preferred embodiment. During this interval, syringe 5 is sanitized by UV-C light. The light from UV-C bulb 25 is green because it is a sign of “working/going” but also is able to be seen under black light which is important for drug users in countries that utilize black lights to prevent syringe use.

While only a few exemplary embodiments are hereinabove described, those knowledgeable in this science will easily appreciate that many modifications are possible in the exemplary embodiments without materially departing from the novel and unique advantages of this disclosure. Accordingly, all such modifications and alternative are intended to be included within the scope of the invention as defined in the following claims. Those skilled in this science should recognize that such modifications and equivalent constructions or methods do not depart from the spirit and scope of the present disclosure, and that they may make various changes, substitutions, and alterations herein without departing from the spirit and scope of the present disclosure.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. A portable syringe sanitizer kit comprising:
a) a longitudinal housing having a sanitization area, a compartment for retaining at least one source of electricity, and means for retaining a needle, barrel, and plunger of a syringe;
b) a source of UV-C light disposed in said housing, said source of UV-C light being electrically connected to said at least one source of electricity; and
c) a switch operatively connected to said at least one source of electricity and to said source of UV-C light for initiating activation thereof.

2. The portable syringe sanitizer kit in accordance with claim 1, further comprising:
d) a syringe holder tube.

3. The portable syringe sanitizer kit in accordance with claim 1, further comprising:
d) means for retaining a syringe plunger.

4. The portable syringe sanitizer kit in accordance with claim 1, wherein said source of UV-C light is one of the group: an LED device, a laser, an electric arc, a xenon bulb, a halogen bulb, an excimer bulb, and a mercury vapor lamp.

5. The portable syringe sanitizer kit in accordance with claim 1, wherein said source of UV-C light generates light having a wavelength of between approximately 200 nm and 280 nm.

6. The portable syringe sanitizer kit in accordance with claim 1, further comprising:
d) an eyeglass case in which is mounted said housing.

7. The portable syringe sanitizer kit in accordance with claim 1, wherein said source of electricity is at least one battery.

8. The portable syringe sanitizer kit in accordance with claim 1, further comprising:
d) a sterilization unit mounted in said sterilization area for receiving at least a portion of a syringe to be sanitized.

9. The portable syringe sanitizer kit in accordance with claim 1, further comprising:
d) a timing device for de-energizing said source of UV-C light after a predetermined interval of time.

10. The portable syringe sanitizer kit in accordance with claim 1, further comprising:
d) a work light operatively connected to said at least one source of electricity and to said switch.

11. A portable syringe sanitizer kit comprising:
a) a longitudinal housing having a sterilization unit, a compartment for retaining at least one source of electricity, and means for retaining a needle, barrel, and plunger of a syringe; and
b) a source of germicidal radiation disposed in said housing, said source of germicidal radiation being electrically connected to said at least one source of electricity.

12. The portable syringe sanitizer kit in accordance with claim 11, further comprising:
c) a syringe holder tube.

13. The portable syringe sanitizer kit in accordance with claim 11, further comprising:
c) means for retaining a syringe plunger.

14. The portable syringe sanitizer kit in accordance with claim 11, wherein said source of germicidal radiation is one of the group: an LED device, a laser, an electric arc, a xenon bulb, a halogen bulb, an excimer bulb, and a mercury vapor lamp.

15. The portable syringe sanitizer kit in accordance with claim 11, wherein said source of germicidal radiation generates light having a wavelength of between approximately 200 nm and 280 nm.

16. The portable syringe sanitizer kit in accordance with claim 11, further comprising:
c) an eyeglass case in which is mounted said housing.

17. The portable syringe sanitizer kit in accordance with claim 11, wherein said source of electricity is at least one battery.

18. The portable syringe sanitizer kit in accordance with claim 11, wherein said sterilization unit receives at least a portion of a syringe to be sanitized.

19. The portable syringe sanitizer kit in accordance with claim 11, further comprising:
c) a timing device for de-energizing said source of germicidal radiation after a predetermined interval of time.

20. The portable syringe sanitizer kit in accordance with claim 11, further comprising:
c) a work light operatively connected to said at least one source of electricity.
